(19) United States
(12) Patent Application Publication
(10) Pub. No.: US 2008/0317873 A1
(43) Pub. Date: Dec. 25, 2008

(54) Pain Relief Compositions

(75) Inventors: Robert G. Schmit, Glenview, IL (US); Matthew W. Zoeller, Chicago, IL (US)

Correspondence Address: FITCH EVEN TABIN AND FLANNERY, 120 SOUTH LA SALLE STREET, SUITE 1600, CHICAGO, IL, 60603-3406, US

(73) Assignee: EPSOM-IT, INC., Chicago, IL (US)

(21) Appl. No.: 12/199,587

(22) Filed: Aug. 27, 2008

Related U.S. Application Data
(62) Division of application No. 11/473,671, filed on Jun. 23, 2006.
(60) Provisional application No. 60/693,297, filed on Jun. 23, 2005.

(51) Int. Cl. A61K 33/04; A61P 25/00

(52) U.S. Cl. 424/709

(57) ABSTRACT

A pain relieving composition for topical application is provided that includes magnesium sulfate (Epsom salts). The composition provides magnesium sulfate in an amount effective for providing pain relief. The composition has a viscosity effective for allowing topical application of the composition to an afflicted area where the magnesium sulfate is absorbed through the skin to provide pain relief. The composition provides the novel benefits of being portable, easily dispensed, and provides a higher concentration of applied magnesium sulfate than the traditional dispensing method of soaking solutions.

This application claims the benefit of U.S. Provisional Application Ser. No. 60/693,297, filed on Jun. 23, 2005. The present invention relates to the field of topical pain relief compositions. More particularly, a composition capable of topical application is provided that includes magnesium sulfate (Epsom salts) in an amount effective for providing pain relief for aching joints and muscles. This invention also relates to a method of delivery of an Epsom salt composition so that it can be applied to the skin and the Epsom salt can be absorbed through the skin to provide pain relief.

### BACKGROUND

People have long enjoyed the ameliorating effects of Epsom salts on their aching joints and muscles. The usual method of gaining these benefits is through dissolving Epsom salts in a bath and soaking the afflicted area. This is inconvenient in that the individual is limited to a stationary position while soaking the afflicted area in the bath. Moreover, treatments using Epsom salts are not very portable and tend to be limited to use in the home.

Accordingly, the pain relief composition of the present invention represents a significant improvement over prior Epsom salt treatments. The composition of the present invention provides the added benefit of affording the user a greater concentration of Epsom salt activity in a topical delivery form comprising 10 to 50 weight percent magnesium sulfate compared to traditional dissolved Epsom salt soaking preparations where the concentration as applied is usually in the range of 0.01 to 3.0 weight percent. Thus, the pain relief composition of the invention is portable and convenient for the consumer to use and does not require the user to remain stationary during treatment. The composition of the invention provides convenient therapy for muscle aches, arthritis pain, and joint stiffness.

### SUMMARY

An Epsom salt composition is provided that has a viscosity effective for allowing its topical application on an area in need of pain relief. This composition provides the same benefits as an Epsom bath but in a convenient topical form. The composition may be a lotion, foam, paste, body rub, cream, gel, serum, stick-type solid, or sprayable solution which includes Epsom salts which, when applied, leaves a film of Epsom salts on the user's skin that are readily absorbed through the skin. Additionally, it is contemplated that the composition may be applied to the user's skin by hand, using an applicator, or by spray. The Epsom salts penetrate the skin as would an Epsom salt bath. Thus, the user can obtain pain relief associated with Epsom salts without the inconvenience of Epsom salt baths.

In one aspect of the invention, an analgesic composition is provided that includes 15 to about 55 weight percent magnesium sulfate, about 1 to about 5 weight percent carrier, about 23 to about 33 weight percent surfactant, about 0.5 to about 2 weight percent salicylate, and about 0.01 to about 0.1 weight percent capsicum. The composition has a viscosity of at least about 4000 cps at a temperature of 25° C., preferably about 4000 cps to about 20,000 cps at 25° C.

In one aspect of the invention, the analgesic composition is produced by mixing silicone surfactant, salicylate, and capsicum to form a silicone mixture. In a separate vessel, magnesium sulfate, carrier, and water are mixed to form a magnesium sulfate mixture. The silicone mixture is then blended with the magnesium sulfate mixture over about 30 minutes to form the analgesic composition. The analgesic composition is then further blended for about 60 to 120 minutes. Finally, the composition passes through a homogenizer, colloid mill, or sheer pump en route to a holding tank.

In another aspect of the invention, an analgesic composition is provided that includes 10 to about 50 weight percent magnesium sulfate, about 2 to about 25 weight percent carrier, about 0.5 to about 5.0 weight percent surfactant, about 0.5 to about 2 weight percent methyl salicylate, and about 0.01 to about 0.1 weight percent capsicum. The composition has a viscosity of at least about 4000 cps at a temperature of 25° C., preferably about 4000 cps to about 16,000 cps at 25° C.

In another aspect of the invention, the analgesic composition is produced by mixing surfactant, water-insoluble carrier, methyl salicylate, and capsicum to form an anhydrous mixture. In a separate container, magnesium sulfate, water-soluble carrier, and water are mixed to form an aqueous magnesium sulfate solution. The anhydrous mixture is slowly combined with the magnesium sulfate solution at moderate temperature over at least about 30 minutes to form the analgesic composition. The analgesic composition is then homogenized.

### DETAILED DESCRIPTION

The present invention provides a pain relief composition comprising Epsom salts (magnesium sulfate). The pain relief composition of the invention may be prepared in a variety of formulations, including lotion, gel, cream, paste, foam, body rub, serum, stick-type solid, sprayable solution, or the like for convenient application by the user. The pain relief composition of the invention is readily absorbed by the skin to provide relief from muscle aches, arthritis pain, and joint stiffness. The composition of the present invention provides a greater concentration of Epsom salt activity in a topical delivery form than previously provided in traditional dissolved Epsom salt soaking preparations.

All weight percents for ingredients described herein are based on the total weight of the composition unless specified otherwise.

The magnesium sulfate of the composition of the invention provides ameliorating pain relief to aching joints and muscles. The magnesium sulfate can be obtained from commercially available Epsom salts. In an important aspect of the invention, the magnesium sulfate is USP grade.

Any pharmaceutically acceptable carrier for topical application may be utilized in the present application. The carrier must provide a viscosity effective for maintaining the composition on the desired site on the body during and after application. For example, a composition with a low viscosity could run off the application site and a composition with too high a viscosity could be difficult to apply. The compositions of the present invention may include about 5.0 to about 25.0 weight percent water-insoluble carrier selected from the group consisting of the following: hydrophobic petroleum distillates such as mineral oil, petrolatum, or ceresin; silicone derivatives such as cyclomethicone and dimethicone; emollient alkyl or aryl esters of fatty alcohols; vegetable oils and their hydrogenated derivatives; or mixtures thereof. The compositions of the present invention may further include about 1.0 to about 5.0 weight percent water-soluble carrier selected from the group consisting of glycerin, propylene glycol, other polyhydric alcohols, or mixtures thereof.

Surfactants useful in the present invention are effective for aiding in emulsification. Preferably, the surfactant is selected from group comprising alkyl-modified ether adducts of dimethicone, hydrophillically modified alkyl fatty acid esters of polyols, or mixtures thereof.

The composition of the invention further includes methyl salicylate. While not intending to be bound by any theory, methyl salicylate may provide an additional analgesic effect. The methyl salicylate may also provide the composition with a desired fragrance.

The composition may further include antimicrobials. Compositions used on the skin should be protected against the growth of potentially harmful microorganisms. The terms antimicrobial and preservative are used interchangeably herein. Any preservative for topical cosmetic or pharmaceutical compositions known in the art may be used with the composition of the present invention. Antimicrobials useful in the invention include parabens (such as methylparaben, ethylparaben, propylparaben, or butylparaben), benzyl alcohol, tetrasodium EDTA (ethylenediaminetetraacetic acid), or any other antimicrobial known in the art. A particularly preferred preservative is available under the tradename GERMABEN® II from Sutton Laboratories (Chatham, N.J.).

The composition further includes capsicum. Capsicum provided by the composition is effective for enhancing the pain relieving benefits. While not intending to be bound by any theory, the capsicum is believed to aid in bringing blood to the surface of the skin which may enhance the pain relief function. Further, the capsicum may also create a warm feeling upon application of the composition, which may enhance the pain relief function. Preferably, the capsicum is capsicum oleoresin. Preferably, the capsicum oleoresin is of the grade 500,000 to 1 MM Scoville Heat Units (SHU).

Ancillary ingredients, such as fragrance, colorants, tocopherol acetate (Vitamin E), aloe vera, emollients, moisturizing agents, or the like may optionally be included in the composition of the present invention. The composition may include about 0.01 to about 2.0 weight percent of these optional ingredients. These optional ingredients should be non-irritating to the skin.

In an important aspect of the invention, an analgesic composition is provided that includes about 15 to about 55 weight percent magnesium sulfate (preferably about 15 to about 25 weight percent), about 1 to about 5 weight percent carrier (preferably about 1.5 to about 2.5 weight percent, more preferably the carrier oil is glycerin), about 23 to about 33 weight percent surfactant, about 0.01 to about 0.1 weight percent capsicum (preferably about 0.025 to about 0.075 weight percent), about 0.5 to about 2 weight percent methyl salicylate (preferably about 0.75 to about 1.25 weight percent), and about 35 to about 65 weight percent water. The composition may further include about 0.1 to about 1 weight percent antimicrobial (preferably about 0.25 to about 0.75 weight percent). Preferably, the composition has a viscosity of at least about 4000 cps at 25° C. and a viscosity of no more than about 16,000 cps at 25° C.

In another important aspect of the invention, an analgesic composition is provided that includes 10 to about 50 weight percent magnesium sulfate (preferably about 10 to about 30 weight percent), about 2 to about 25 weight percent carrier (preferably 5 to 15 weight percent), about 0.5 to about 5.0 weight percent surfactant, about 0.5 to about 2 weight percent methyl salicylate, about 0.01 to about 0.2 weight percent capsicum, and about 35 to about 65 weight percent water. Preferably, the surfactant is an alkyl-modified ether adduct of dimethicone. The composition may further include about 0.1 to about 1.0 weight percent, preferably 0.3 to about 1.0 weight percent, water-soluble antimicrobials. Preferably, the composition includes about 0.5 to 3.0 weight percent surfactant from the class of alkyl-modified ether adducts of dimethicone and may further include about 0.1 to about 2.0 weight percent of a second surfactant from a class of hydrophillically modified alkyl fatty acid esters of polyols.

The composition of the present invention is formulated for use on the skin, preferably applied as needed, to achieve the desired pain relief. The composition of the present invention may be provided in a variety of formulations and viscosities, such as in a lotion, cream, foam, paste, gel, body rub, sprayable solution, serum, stick-type solid, or the like. Depending on the form of the composition, the composition may be rubbed, poured, or sprayed onto the skin. The composition is applied to the skin on an as needed basis for as long as the pain relieving effect is desired.

In another aspect of the invention, the composition has may be provided in a variety of formulations with the following viscosities: about 20 to about 4000 cps for sprayable liquids, serums, and the like; about 4000 to about 20,000 cps for lotions, liniments, and the like; about 20,000 to about 300,000 cps for creams, gels, pastes, ointments, and the like; and sedentary for stick solids.

It is further contemplated that the composition of the present invention may be provided in combination with a variety of other skin treatment compositions, such as medicated lotions, suntan lotions, moisturizers, anti-aging compositions, the like, or mixtures thereof.

### Preparation of the Composition

In one aspect of the invention, an analgesic composition of the present invention is produced by mixing surfactant, salicylate, and capsicum to form a surfactant mixture. In a separate container, magnesium sulfate, carrier, and water are mixed to form a magnesium sulfate mixture. The surfactant mixture and the magnesium sulfate mixture are slowly combined at slow mixer speed, such as about 120 to about 600 rpm, over at least about 30 minutes to form the analgesic composition. If the magnesium sulfate mixture and the surfactant mixture are combined too quickly, the resulting emulsion may exhibit instability. After all of the magnesium sulfate mixture has been added to the silicone mixture, the resulting mixture is mixed for about 60 to about 120 minutes to form the pain relieving composition, which is then homogenized for at least 30 minutes at about 1200 to about 3500 rpm if an in-process homogenizer is employed or a single pass at about 1700 to 4000 rpm at a gap of 0.025 to 0.06″ if a stationary rotor-stator type of homogenizer is used.

In another aspect of the invention, an analgesic composition of the present invention is produced by mixing surfactant, water-insoluble carrier, methyl salicylate, and capsicum to form an anhydrous mixture. In a separate container, magnesium sulfate, waters soluble carrier, and water are mixed to form an aqueous magnesium sulfate solution. If the magnesium sulfate solution and the anhydrous mixture are combined too quickly, the resulting emulsion may exhibit instability. The magnesium sulfate solution and the anhydrous mixture are combined very slowly under slow mixing (over a period of 30 to 60 minutes at a mixer speed of about 120 to about 600 rpm) to the homogenous mixture. After the magnesium sulfate solution and homogenous mixture have been combined, the resulting mixture is mixed for about 60 to about 120 minutes to form the pain relieving composition, which is then homogenized for at least 30 minutes at about 1200 to about 3500 rpm if an in-process homogenizer is employed or a single pass at about 1700 to 4000 rpm at a gap of 0.025 to 0.06″ if a stationary rotor-stator type of homogenizer is used.

Numerous modifications and variations in practice of the invention are expected to occur to those skilled in the art upon consideration of the foregoing detailed description of the invention. Consequently, such modifications and variations are intended to be included within the scope of the following claims.

1-18. (canceled)

19. A method of producing a pain relieving composition comprising:
mixing a surfactant, water-insoluble carrier, methyl salicylate, and capsicum to form an anhydrous mixture;
mixing water, magnesium sulfate, and a water-soluble carrier to form an aqueous solution; and
blending the anhydrous mixture with the aqueous solution to form the pain relieving composition.

20. The method of claim 19 wherein the water-insoluble carrier is selected from the group consisting of petroleum distillates, including mineral oil, petrolatum, or ceresin, silicone derivatives including cyclomethicone or dimethicone, emollient alkyl esters of fatty alcohols, vegetable oils and their hydrogenated derivatives, or mixtures thereof.

21. The method of claim 19 comprising from about 5 to about 25 weight percent water-insoluble carrier.

22. The method of claim 19 comprising from about 1.0 to about 5.0 weight percent water-soluble carrier.

23. The method of claim 19 wherein the water-soluble carrier is selected from the group consisting of glycerin, propylene glycol, other polyhydric alcohols, or mixtures thereof.

24. The method of claim 19 wherein the surfactant is selected from the group consisting of alkyl-modified ether adducts of dimethicone, hydrophillically modified alkyl fatty acid esters of polyols, or mixtures thereof.

25. The method of claim 19 comprising from about 0.5 to about 5.0 weight percent surfactant.

26. The method of claim 19 wherein the capsicum is capsicum oleoresin.

27. The method of claim 19 comprising from about 0.01 to about 0.2 weight percent capsicum.

28. The method of claim 19 comprising from about 0.5 to about 2.0 weight percent methyl salicylate.

29. The method of claim 19 further comprising an antimicrobial.

30. The method of claim 29 comprising from about 0.1 to about 1 weight percent antimicrobial.

31. The method of claim 19 wherein the anhydrous mixture and aqueous solution are combined over at least about 30 minutes, wherein the pain relieving composition is then blended for about 60 to about 120 minutes, and wherein the pain relieving composition is homogenized after blending.
